(19) United States
(12) Patent Application Publication
(10) Pub. No.: US 2022/0280606 A1
(43) Pub. Date: Sep. 8, 2022

(54) METHOD OF PREPARING A DE-FIBRINATED PLATELET LYSATE, AND USES OF SAID METHOD

(71) Applicants: Abdalla S AWIDI, Cambridge, MA (US); Hanan JAFAR, Amman (JO)

(72) Inventors: Abdalla S AWIDI, Cambridge, MA (US); Hanan JAFAR, Amman (JO)

(21) Appl. No.: 17/637,406

(22) PCT Filed: Aug. 24, 2020
(86) PCT No.: PCT/US2020/047595

§ 371 Date: Feb. 22, 2022

Related U.S. Application Data
(60) Provisional application No. 62/890,252, filed on Aug. 22, 2019.

(51) Int. Cl. A61K 38/18; A61K 35/16; A61K 35/19; A61K 31/728; A61K 45/06; A61K 47/14; A61K 38/48; A61K 47/02; A61K 9/00; C07K 1/32

(52) U.S. Cl. CPC A61K 38/1808; A61K 35/16; A61K 35/19; A61K 31/728; A61K 45/06; A61K 47/14; A61K 38/4833; A61K 47/02; A61K 9/0014; A61K 38/1866; A61K 38/1825; A61K 38/1841; A61K 38/1858; C07K 1/32

(57) ABSTRACT

This invention relates to the methods of preparation of a growth factor concentrate derived from platelet lysate and modified to be devoid of fibrinogen. Moreover, the present invention concerns therapeutic applications of said growth factor concentrate to enhance mitogenic, osteogenic, chondrogenic and angiogenic abilities. The disclosed preparation can be used immediately, or stored frozen until use, or lyophilized, or mixed with pharmaceutical formulation, such as biodegradable polymer or other delivery vehicles, to make products.

### BACKGROUND OF THE INVENTION

Platelets, or thrombocytes, are cytoplasmic fragments of megakaryocytes circulating in an intact inactivated form. Their primary function is to aggregate on the site of injury and form a “plug”, thereby contributing to hemostasis. Additionally, activated platelets also release several growth factors, including but not limited to epidermal growth factor (EGF), transforming growth factor beta (TGF-β), fibroblast growth factor (FGF), platelet derived growth factor (PDGF), insulin like growth factor (ILGF), vascular endothelial growth factor (VEGF) and basic fibroblast growth factor (_{b}-FGF). These growth factors play important roles in tissue regeneration, as they stimulate the body's healing process. Accordingly, the use of platelet rich plasma (PRP) has been advocated as a potential therapeutic option for some medical conditions.

Several medical conditions that involve delayed healing or tissue loss are refractory to classical treatments. Examples include, but are not limited to, long standing ulcers, peripheral vascular disease, persistent corneal ulcers, joint cartilage degeneration, cardiac abnormalities, mucositis, erectile dysfunction, age-related creases, alopecia, alveolar bone osteonecrosis, dental pulp inflammations, and others. In many of these diseases, platelet products such as PRP, have been shown to exert positive curative or restorative effects.

PRP, as indicated by the name, is a formulation derived from blood, in which the concentration of platelets is higher than the baseline of normal count, and the platelets are suspended in a small volume of plasma. One drawback of this technique is the lack of standardization, owed to the fact that platelets are not usually counted and therefore their “concentration” is not established. Additionally, inter-personal differences in multiple platelet parameters are noted and are correlated to varying concentrations of growth factors. These sources of variations lead to variable outcomes among individuals.

Several growth factors released from platelets have been also been shown to enhance in vitro cell growth. Traditionally, cells in culture, including stem cells, are expanded in media supplemented with animal sera such as fetal bovine serum (FBS). However, due to issues of disease transmission and antigenicity, focus has been shifted to the introduction of xeno-free products and methodology. Additionally, legal authorities worldwide are requiring the application of good manufacturing practice to cellular products intended for therapeutic use. Recently, platelet lysate has been suggested as a substitute for FBS.

Platelets are collected routinely for therapeutic purposes. Several steps are required before they can be used as tissue culture supplements. First, they should be concentrated to standardize their numbers and prevent inter-batch variability. Platelets should be activated following the concentration process. This is customarily done through the addition of thrombin and calcium chloride, consequently de-granulating platelets and releasing their contents. The downside of this method is the use of bovine thrombin, potentially carrying the threat of transmissible diseases or immune reactions. Accordingly, a new preparation was developed through lysing platelets utilizing freeze/thaw cycles. This was termed Platelet Lysate (PL). Freezing, however, has been reported to cause the formation of a fibrin clot, which would trap several of the growth factors in the lysate. The fact that fibrinogen (a soluble protein present in blood plasma, from which fibrin is produced by the action of the enzyme thrombin) is still a component of the product requires the use of anticoagulants in cell culture media to prevent clotting. These anticoagulants are mostly of animal origin.

Typically, heparin and calcium salt are applied to initiate the clotting cascade. (See, e.g., U.S. Pat. No. 9,688,952 (Copland and Galipeau, 2017), however, this method is calcium dose-dependent. Additionally, calcium concentration in the product would be altered from physiologic state. The use of heparin to control clotting would cause the final product to be more expensive. The physical removal of the clot also depletes a significant amount of the overall volume of product.

There is a need in the art for an improved method for removing fibrinogen from plasma to create a platelet rich lysate free of fibrinogen that preserves the growth factors within the lysate.

### Definitions

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference; thus, the inclusion of such definitions herein should not be construed to represent a substantial difference over what is generally understood in the art.

Within the framework of the present description and in the subsequent claims, except where otherwise indicated, all numbers expressing amounts, quantities, percentages, and so forth, are to be understood as being preceded in all instances by the term “about”. As used herein, the term “about” is defined as ±5%. Also, all ranges of numerical entities include all the possible combinations of the maximum and minimum numerical values and all the possible intermediate ranges therein, in addition to those specifically indicated hereafter.

“Therapeutic applications” include any process in which it is advantageous to enhance mitogenic, osteogenic, chondrogenic or angiogenic activities in the region of application to a patient in need of treatment.

The term “pharmaceutically acceptable salts or derivatives” as used herein refers to those salts or derivatives which possess the biological effectiveness and properties of the salified or derivatized compound and which do not produce adverse reactions when administered to a mammal, preferably a human. The pharmaceutically acceptable salts may be inorganic or organic salts; examples of pharmaceutically acceptable salts include but are not limited to: carbonate, hydrochloride, hydrobromide, sulphate; hydrogen sulphate; citrate, maleate, fumarate, tifluoroacetate, 2-naphthalenesulphonate, and para-toluenesulphonate. Further information on pharmaceutically acceptable salts can be found in Handbook of pharmaceutical salts, P. Stahl, C. Wermuth, WILEY-VCH, 127-133, 2008, herein incorporated by reference. The pharmaceutically acceptable derivatives include the esters, the ethers and the N-oxides.

The term “pharmaceutically-acceptable excipient” as used herein refers to a substance devoid of any pharmacological effect of its own and which does not produce adverse reactions when administered to a mammal, preferably a human. Physiologically acceptable excipients are well known in the art and are disclosed, for instance in the Handbook of Pharmaceutical Excipients, sixth edition 2009, herein incorporated by reference. The list of pharmaceutically-acceptable excipients includes, but is not limited to: bulking agents, lyoprotectants, buffering agents, tonicity adjusting agents, collapse temperature modifiers, antioxidants, antimicrobial preservatives, chelating agents, reducing agents, surfactants, complexing and dispersing agents, suspending agents, wetting agents and flocculating agents, viscosity building agents. The list further comprises mannitol, sucrose, lactose, polyethylene glycol, polyvinyl pyrollidone, glycine and combinations thereof.

The term “allogenic” as used herein refers to cells, cell fragments, or plasma from one or more donors, obtained by collection from said donors, for the purpose of infusion or other introduction into a patient in need of such. This includes the cells, cell fragments, or plasma from multiple suitable donors which are pooled together.

The term “autologous” as used herein refers to cells, cell fragments, or plasma from a patient which are collected and then re-infused or re-introduced into the same patient.

The term “platelet” as used herein refers to fragments of megakaryocyte cells of the bone marrow found in normal human circulation. The word “thrombocytes” may also be used for this definition.

The term “lysate” as used herein refers to a fluid containing the contents of lysed cells or cell fragments. “Lysed” cells or fragments have their membrane integrity disrupted, either by enzymatic or physical means, resulting in their contents being released into the lysate.

The term “lyophilization” as used herein refers to a low temperature dehydration process which involves freezing the product, lowering pressure, then removing the ice by sublimation. Lyophilized products of the invention may be formed as powders or combined with pharmaceutically acceptable excipients, as listed above.

The term “apheresis” as used herein refers to a method in which the blood of a subject is removed, one particular constituent of the blood (such as platelets) is separated out, and the remained is returned to the subject's circulation.

The term “mitogenic” as used herein refers to a chemical or biological substance that promotes cell division when exposed to a living cell.

The term “osteogenic” as used herein refers to a chemical or biological substance that promotes the deposit of bone material by osteoblasts.

The term “chondrogenic” as used herein refers to a chemical or biological substance that promotes the creation of cartilage.

The term “angiogenic” as used herein refers to a chemical or biological substance that promotes the creation of new blood vessels.

The term “wound” as used herein refers to any type of injury in which the skin is torn, cut, punctured, or otherwise damaged. The damage may be caused by accidental trauma, deliberate action (e.g. a surgeon's incision with a scalpel) or as a result of disease.

The term “blood sample” as used herein refers to a set amount of human blood usually extracted from the vein in the arm of a human volunteer. It may also refer to a set amount of human blood obtained from a blood bank or other entity in the business of collecting blood from healthy, human volunteers.

The term “fibrinogen-selective binding agent” as used herein refers to a ligand or other molecule that binds more preferentially to fibrinogen than to other proteins or molecules found in human plasma.

### SUMMARY OF THE INVENTION

The present invention provides for a method for removing fibrinogen from plasma.

The present invention further provides a process for the preparation of a growth factor concentrate or platelet lysate derived from human platelets that is depleted of fibrinogen while containing an ideal growth factor concentrate. In a preferred embodiment, the platelets are pooled allogenic O negative platelets. The terms “platelet lysate” and “growth factor concentrate” are used interchangeably in this context.

In another embodiment, the invention provides a method for preparing an intra-dermally, sub-dermally, intra-articularly, intra-pulpally or topically administered growth factor concentrate derived from human platelets and depleted of fibrinogen.

In a further embodiment, the growth factor concentrate or platelet lysate of the invention is used as a therapeutic application. In yet a further embodiment, the growth factor concentrate of the invention is applied locally to the area or cells of a patient in need of treatment thereof.

In an embodiment, the growth factor concentrate or platelet lysate of the invention is prepared or administered as a pharmaceutical composition. In various embodiments, the pharmaceutical composition may include a pharmaceutically-acceptable excipient, salt, or derivative. In various embodiments, the pharmaceutical composition may include additional components, such as blood, saline, silver nanoparticles, hyaluronic acid, immuno-modulatory peptides, growth factors, hormones, antibiotics, recombinant receptors, carriers or combinations thereof.

Pharmaceutical compositions of the invention may be in the form of a cream, gel, aqueous solution, spray-aerosol, or transdermal patch. In various embodiments, the pharmaceutical composition further includes a slow-release biodegradable delivery system.

In another aspect, the invention relates to the inclusion of said growth factor concentrate in culture media for the purpose of growing cells. More particularly, for the purpose of growing stem cells, both for research purposes, as well as potential clinical, therapeutic, or diagnostic applications of the cells.

In another embodiment of the present invention, a dehydrated composition is provided comprising lyophilized platelet lysate among lyophilization excipients, that can be reconstituted at point of care. In a further embodiment, one or more pharmaceutically-acceptable excipient, salt, or derivatives is added for the lyophilization process and is lyophilized.

The lyophilized composition may be reconstituted using a biocompatible aqueous solution. In an embodiment, the biocompatible aqueous solution is pharmaceutical-grade saline or Ringer's solution.

### BRIEF DESCRIPTION OF THE DRAWINGS

The following detailed description of the invention reference is made to the accompanying drawings which form a part hereof, and in which are shown, by way of illustration, specific embodiments in which the invention may be practiced. These embodiments are described in sufficient detail to enable those skilled in the art to practice the invention. Other embodiments may be utilized, and structural and logical changes may be made, without departing from the scope of the present invention.

FIG. 1 shows a flow diagram for a process of generating an autologous growth factor concentrate depleted of fibrinogen;

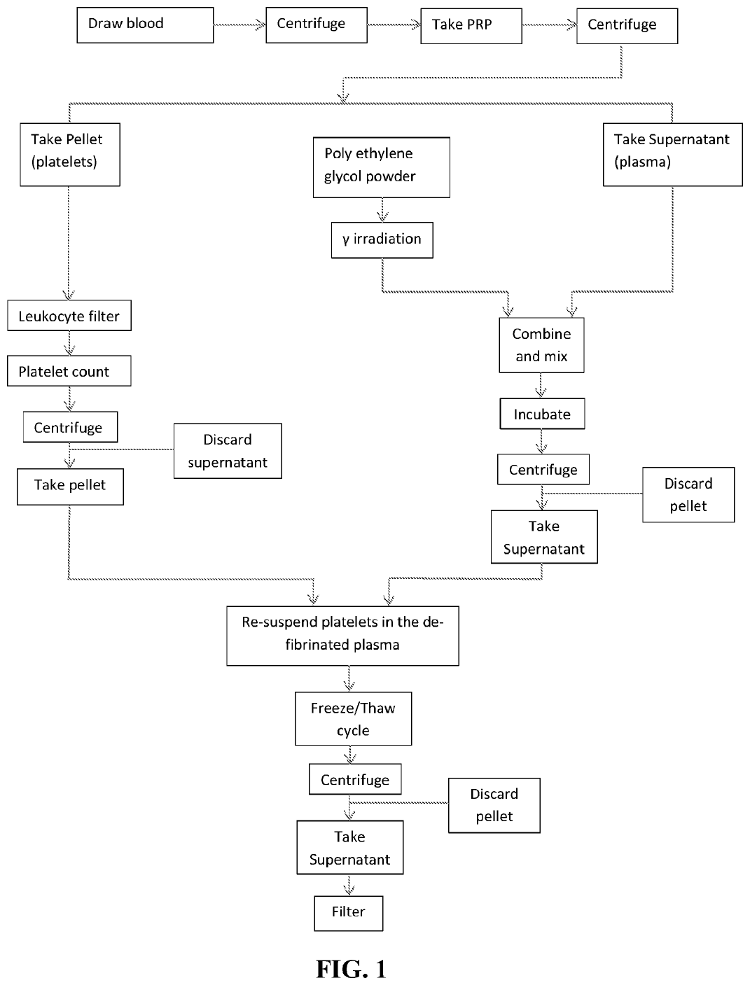

FIG. 2 illustrates a flow diagram for a preferred process of generating an allogenic growth factor concentrate depleted of fibrinogen;

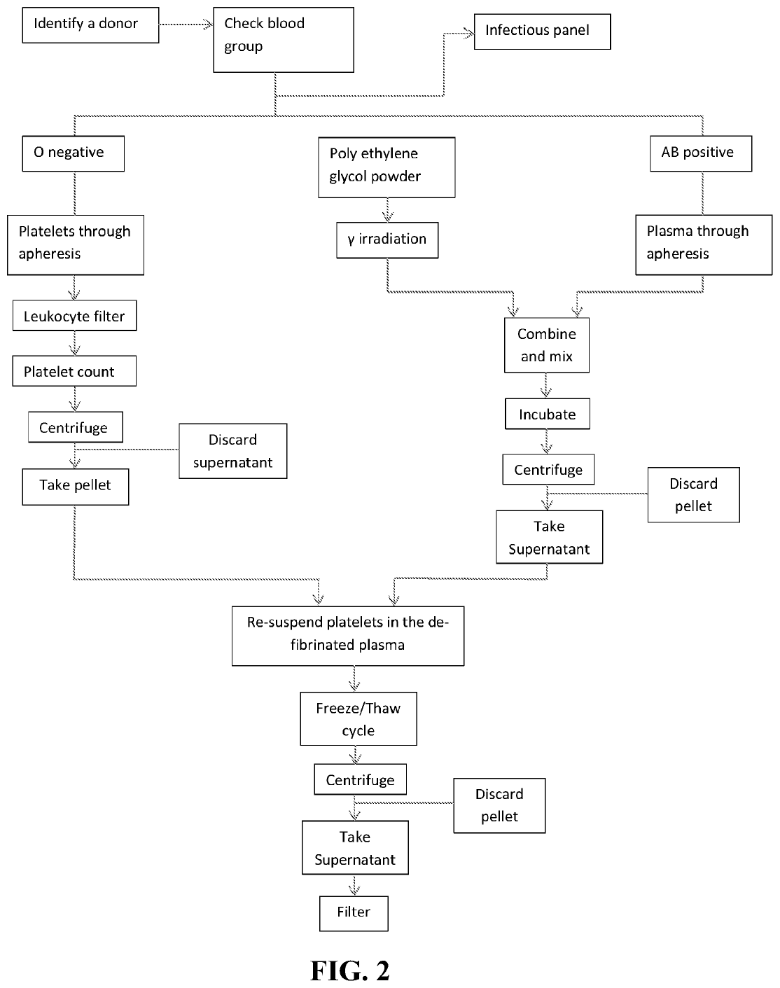

FIG. 3 illustrates a flow diagram for the process of generating a lyophilized growth factor concentrate depleted of fibrinogen;

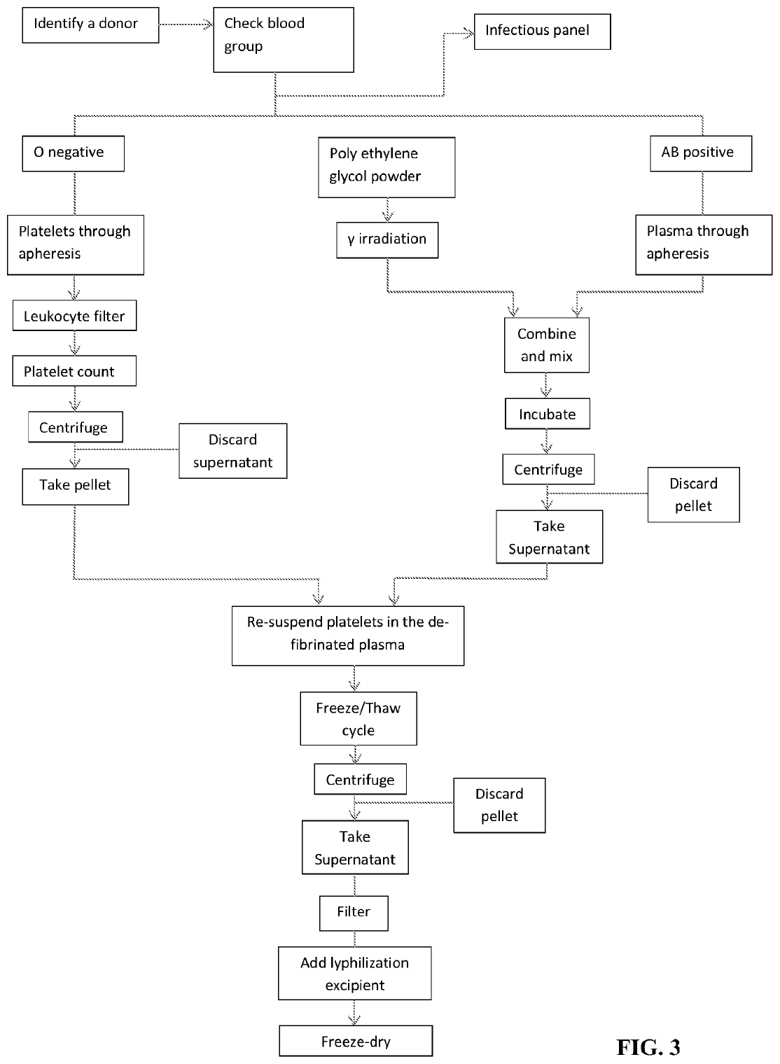

FIG. 4 shows graphical data of levels of fibrinogen and other clotting factors in the final product. Bar graphs are provided for Factor I, Factor II, Factor IV, Factor V, Factor VII, Factor VIII, Factor IX, Factor X, Factor XI and Factor XII, respectively. The “left bar” represents the de-fibrinated platelet lysate prepared according to the teachings of the instant application whereas the “right bar” represents the naturally-occurring platelet lysate for the respective factors. Factors I, VIII, IX and XI were completely de-fibrinated and, as such, do not have a “left bar”; and

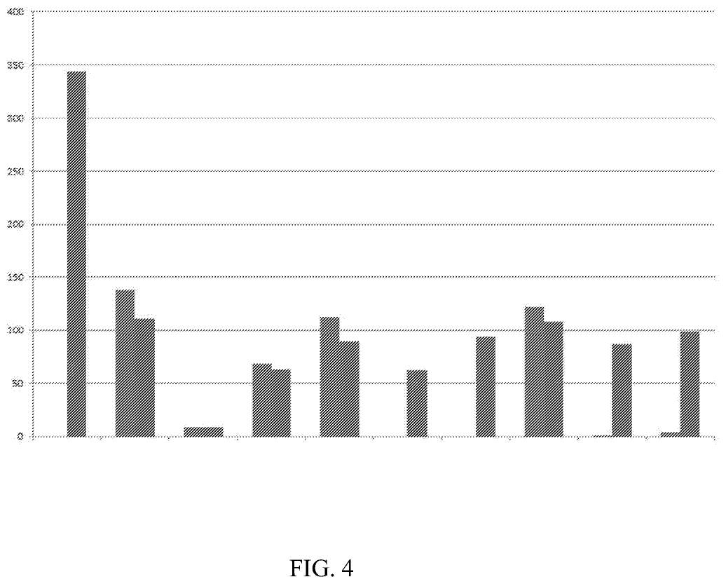

FIG. 5 shows graphical data of levels of desirable growth factors in the final product, such as PDGF, TGF-β and EGF in pg/10⁶ platelets measured by ELISA. Bar graphs are provided for TGF-β, PDGF-BB, PDGF-AB, FGF, EGF, TGF-α, VEGF-C and VEGF-A, respectively. The “left bar” represents the naturally-occurring platelet lysate whereas the “right bar” represents the de-fibrinated platelet lysate prepared according to the teachings of the instant application. The measured values for each are as follows:

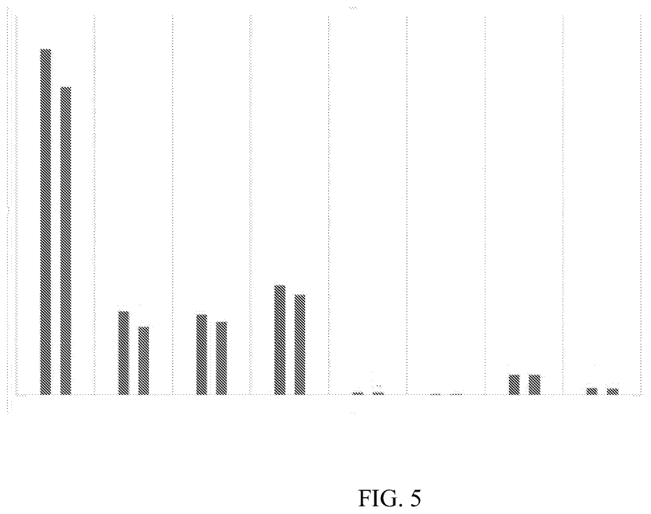

| Growth | Measured Naturally-Occurring | Measured De-fibrinated |
|---|---|---|
| Factor | Platelet Lysate, i.e. “left bar” | Platelet Lysate, i.e. “right bar” |
| TGF-β | 182.3 | 162.4 |
| PDGF-BB | 44.1 | 35.8 |
| PDGF-AB | 42.3 | 38.6 |
| FGF | 57.6 | 52.4 |
| EGF | 1.56 | 1.328 |
| TGF-α | 0.696 | 0.36 |
| VEGF-C | 10.815 | 10.48 |
| VEGF-A | 3.648 | 3.256 |

### DETAILED DESCRIPTION OF THE INVENTION

The present invention details a new methodology for preparing a platelet lysate depleted of fibrinogen.

For convenience, certain terms employed in the specification, examples and appended claims are collected here. These definitions should be read in light of the disclosure and understood as by a person of skill in the art.

As used herein, the terms “comprises.” “comprising.” “includes.” “including.” “has” having or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, ‘or’ refers to an inclusive or and not to an exclusive or. For example, a condition A or B is satisfied by any one of the following: A is true (or present) and B is false (or not present), A is false (or not present) and B is true (or present), and both A and B are true (or present). Also, use of the “a” or “an” are employed to describe elements and components of the invention. This is done merely for convenience and to give a general sense of the invention. The term “and/or” as used herein is defined as the possibility of having one or the other or both. For example, “A and/or B” provides for the scenarios of having just A or just B or a combination of A and B. If the claim reads A and/or B and/or C, the composition may include A alone, B alone, C alone, A and B but not C, B and C but not A, A and C but not B or all three A, B and C as components. This description should be read to include one or at least one and the singular also includes the plural unless it is obvious that it is meant otherwise. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting. In the following description, numerous specific details are provided, such as the identification of various system components, to provide an understanding of embodiments of the invention. One skilled in the art will recognize, however, that embodiments of the invention can be practiced without one or more of the specific details, or with other methods, components, materials, etc. In still other instances, well-known structures, materials, or operations are not shown or described in detail to avoid obscuring aspects of various embodiments of the invention. Reference throughout this specification to “one embodiment” or “an embodiment’ means that a particular feature, structure, or characteristic described in connection with the embodiment is included in at least one embodiment of the present invention. Thus, the appearance of the phrases “in one embodiment” or “in an embodiment in various places throughout this specification are not necessarily all referring to the same embodiment. Furthermore, the particular features, structures, or characteristics may be combined in any suitable manner in one or more embodiments.

Platelets can be collected via apheresis or from whole blood. Although platelets display ABO antigens, ABO-incompatible platelets are routinely used in transfusions and do not usually present an immediate concern. Therefore, platelets can be used for the same individual from whom they were collected, i.e. autologous, or for others, i.e. allogenic. The same would apply to other platelet products, such as PRP and PL.

Using allogenic platelets to prepare said growth factor concentrate is disclosed since it is more advantageous. They can be obtained from pedigree blood (i.e., individuals screened for transmissible diseases) or from outdated platelets (platelets that have been in storage for a week or more) obtained from blood banks. Preferably, platelets are pooled from many sources.

Platelets contain granules that have mitogenic and angiogenic factors. Lysing platelets will result in freeing these factors, and therefore exploiting their mitogenic and angiogenic effects. This is typically done via freeze/thaw cycles, but is not the only method. Additional contemplated procedures for lysing platelets include, among others, mechanical approaches such as sonication. Further, lysis buffers, which contain hypotonic solutions, can also be used. Freezing techniques include, but are not limited to, placing the cells in an electric refrigeration unit for an extended period of time, snap-freezing, ice bath and liquid nitrogen in complete medium.

In a preferred embodiment, the method of the invention may be used in the preparation of growth factor concentrate wherein fibrinogen is substantially depleted.

The instant invention describes a method by which fibrinogen is removed by adding a material that selectively attaches to fibrinogen and removes it from the product. The material is then removed entirely from the product without affecting any other constituent or initiating any reaction. Said material is disclosed as polyethelene glycol (PEG), but any other materials with similar abilities to adhere to fibrinogen, such as amorphous carbon, diamond like carbon (DLC) or others, can be used. These materials may include polyethers, polymers or oligomers of ethylene oxide.

In certain embodiments, the current invention discloses a method by which plasma is collected from a donor other than the platelet donor for allogenic use. Contemplated advantages include a plasma component free of anti-agglutinins that would potentially cause immune reactions.

In a preferred embodiment, the platelets are collected from donors with O negative blood group, while the plasma component is collected from donors with AB positive blood group.

It is contemplated that said growth factor concentrate disclosed here can be mixed with other pharmaceutical formulations to produce other products with advantageous properties. Such contemplated formulations include, but are not limited to, materials that would produce a cream or ointment for topical applications.

In an embodiment, the growth factor concentrate of the invention is used in therapeutic applications, either intra-dermally, sub-dermally, intra-articularly, intra-pulpally or topically administered. Possible therapeutic applications of the growth factor concentrate of the invention include, but are not limited to:

Treatment of chronic non-healing cutaneous wounds, including but not limited to: ischemic wounds, diabetic wounds, ischemic wounds from atherosclerosis and wounds from arteriolar vasculitis, venous stasis wounds, including post-phlebitic syndrome and post-traumatic venous stasis, pressure sores, including sacral decubitus, ischial decubitus, heel and malleolar decubitus, and pressure sores applied to other areas, and wounds from persisting cutaneous trauma.

Topical treatment of acute wounds, including but not limited to: split thickness wounds, skin graft donor site wounds, abrasions (such as those occurring from the skin contacting a rough surface such as pavement due to an accident on a moving bicycle, scooter, moped, motorcycle, automobile, sled, skateboard, roller skates, skateboard or inline skates), full thickness skin loss, degloving injury, traumatic skin loss, and traumatic skin necrosis. Burns, including but not limited to: split thickness skin graft donor site repair, acceleration of granulation tissue formation and early debridement and grafting of skin, acceleration of re-epithelization of second degree burns, and improvement of cosmetic result in skin grafting by prevention of chronic contracture.

Revascularization of intact skin, including but not limited to: necrobiosis lipoidica diabeticorum radiation induced skin ischemia, and phemphigus vulgaris.

Cosmetic applications, including but not limited to: treatment of androgenetic alopecia, the promotion of hair growth, the reversal of periorbital hyperpigmentation, the renewal of skin growth, the reduction of nasolabial and/or facial wrinkles, the treatment of acne and acne scars, amelioration of tennis elbow, the treatment of diabetic foot ulcers, reduction of fistulas and the reversal of age-related dermatological changes.

In another embodiment, the growth factor concentrate of the invention is used in the treatment of acute surgical wounds, including internal wounds or traumatic wounds. Compositions including the growth factor concentrate of the invention may enhance the rate of normal wound repair by shortening the lag phase. The growth factor concentrate or composition containing such may be applied topically to any surgical wound, either in the skin (dermis or epidermis) or internal organs. The types of wound to be treated include, but are not limited to: liver lacerations, kidney lacerations, splenic lacerations and anastomoses of, for example, the bowel, colon, or biliary tree, atraumatic wounds, atraumatic wounds of the liver and spleen, and intra-abdominal abscesses. For example, when an intra-abdominal abscess is drained percutaneously and the drain is left in place, compositions could be injected through the drain so as to topically apply the composition to the surfaces of the cavity to accelerate repair of that potential space.

In another embodiment, the growth factor concentrate of the invention is used to enhance bone grafts and accelerate soft tissue maturation in aesthetic periodontal surgery.

In another embodiment, the growth factor concentrate of the invention is used in culture media for the purpose of growing cells; more particularly, for the purpose of growing stem cells, both for research purposes, as well as potential clinical, therapeutic, or diagnostic applications of the cells. In various further embodiments, the cell culture systems include, but are not limited to, mesenchymal stem cells of bone marrow origin, adipose tissue origin, umbilical cord origin, placenta origin, dental origins, and others.

### EXEMPLIFICATION

### Example 1

Fifty milliliters whole blood were aseptically collected from an individual in acid citrate dextrose (ACD) anticoagulant. The blood was mixed well with ACD. The anticoagulated blood was centrifuged at 100×g for 10 minutes. No braking was applied. The supernatant, termed hereafter platelet rich plasma (PRP), was carefully transferred into sterile 50 ml conical centrifuge tubes using sterile pipettes under an isolator. Platelet count was performed on the PRP using known methods in the art. PRP was centrifuged at 3300×g for 10 minutes and allowed to stop without brakes. The supernatant, termed hereafter plasma was transferred into sterile 50 ml conical centrifuge tubes and incubated with polyethelene glycol (PEG) overnight. The platelet pellet was stored at 4° C. overnight. The following day, the plasma/PEG mixture was centrifuged at 3300×g for 10 minutes, and the processed plasma was transferred to sterile tubes. Platelets were resuspended in adequate amounts of processed plasma to a final platelet concentration of 1×10⁶. The resuspended platelets were frozen at −80° C. and thawed at 37° C. Freeze/thaw was repeated 3 times, to produce the growth factor concentrate. The final growth factor concentrate was frozen in 5 ml aliquots for storage and subsequent use or lyophilized or immediately used with pharmaceutical formulation to make products. Generally, the plasma volume to be processed is about 5 times lesser than the volume of whole blood collected from the donor.

### Example 2

Plasma was collected via apheresis from a donor with blood group AB (Rh+). Collected plasma was combined and mixed with gamma-irradiated polyethelene glycol (PEG) and incubated overnight. The following day, plasma/PEG mixture was centrifuged at 3300×g for 10 minutes, and the processed plasma transferred to sterile tubes. Platelets were collected via apheresis from a donor with blood group O (Rh−). A platelet count was performed and the collected platelets centrifuged at 3300×g for 10 minutes. The platelet pellet was resuspended in adequate amounts of processed plasma to a final platelet concentration of 1×10⁶ and/or a platelet concentration that is three to eight times greater than native plasma. The resuspended platelets were frozen at −80° C. and thawed at 37° C. Freeze/thaw was repeated 3 times, to produce the growth factor concentrate. The final growth factor concentrate was frozen in 5 ml aliquots for storage and subsequent use or lyophilized or immediately used with pharmaceutical formulation to make products.

### EQUIVALENTS

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

The above discussion is meant to be illustrative of the principle and various embodiments of the present invention. Numerous variations, combinations and modifications will become apparent to those skilled in the art once the above disclosure is fully appreciated. It is intended that the following claims be interpreted to embrace all such variations and modifications.

What is claim is:

1. A method to remove fibrinogen from plasma, comprising the steps of:
collecting a blood sample;
removing the platelets from said blood sample;
adding a fibrinogen-selective binding agent to said platelet-containing solution;
suspending said platelets in a solution;
removing said binding agent with bound fibrinogen from said platelet-containing solution.

2. The method according to claim 1, wherein said blood sample is collected from a human donor or from already collected blood stored in a blood bank.

3. The method according to claim 2, wherein said collected blood is allogenic blood.

4. A method to prepare a growth factor concentrate comprising the steps of:
collecting blood from a human donor;
removing the platelets from said collected blood;
suspending said platelets in a solution;
adding a fibrinogen-selective binding agent to said platelet-containing solution;
removing said binding agent with bound fibrinogen from said platelet-containing solution.

5. The method according to claim 4, wherein said growth factor concentrate may be administered to a patient suffering from a disease, illness, indication or condition in which growth factor concentrate is an accepted treatment for such a disease, illness, indication or condition in need of treatment thereof, wherein said growth factor concentrate is administered to said patient intra-dermally, sub-dermally, intraarticularly, intra-pulpal or topically.

6. A method to prepare a platelet lysate depleted of fibrinogen, comprising the steps of:
collecting blood from a human donor;
removing the platelets from said collected blood;
suspending said platelets in a solution;
adding a fibrinogen-selective binding agent to said platelet-containing solution;
removing said binding agent with bound fibrinogen from said platelet-containing solution.

7. The fibrinogen-depleted platelet lysate according to claim 6, wherein said collected blood is allogenic blood.

8. The fibrinogen-depleted platelet lysate according to claim 6, wherein said collected blood is pedigree blood.

9. The fibrinogen-depleted platelet lysate according to claim 6, wherein said fibrinogen-depleted platelet lysate enhances the mitogenic, osteogenic, chondrogenic or angiogenic activity of the cells of a patient in need of treatment with said fibrinogen-depleted platelet lysate.

10. The fibrinogen-depleted platelet lysate according to claim 9, wherein said cells are located in an area in which said fibrinogen-depleted platelet lysate is administered to a patient in need of treatment thereof.

11. A method of enhancing the mitogenic, osteogenic, chondrogenic or angiogenic activity of the cells of a patient in need of treatment by administering the growth factor concentrate according to claim 4 to said patient.

12. A pharmaceutical composition comprised of the growth factor concentrate according to claim 4.

13. The pharmaceutical composition according to claim 12, further comprising one or more pharmaceutically-acceptable excipients.

14. The pharmaceutical composition according to claim 13, wherein said one or more pharmaceutically-acceptable excipients are selected from the group consisting of bulking agents, lyoprotectants, buffering agents, tonicity adjusting agents, collapse temperature modifiers, antioxidants, antimicrobial preservatives, chelating agents, reducing agents, surfactants, complexing and dispersing agents, suspending agents, wetting agents and flocculating agents, viscosity building agents.

15. The pharmaceutical composition according to claim 12, further comprising additional components selected from the group consisting of blood, saline, silver nanoparticles, hyaluronic acid, immuno-modulatory peptides, growth factors, hormones, antibiotics, recombinant receptors, carriers or combinations thereof.

16. The pharmaceutical composition according to claim 14, wherein said one or more pharmaceutically-acceptable excipient is selected from the group consisting of mannitol, sucrose, lactose, polyethylene glycol, polyvinyl pyrollidone, glycine and combinations thereof.

17. The pharmaceutical composition according to claim 13, wherein the composition is in the form of a cream, gel, aqueous solution, spray-aerosol or transdermal patch.

18. The pharmaceutical composition according to claim 12, wherein said growth factor concentrate is lyophilized.

19. The pharmaceutical composition according to claim 13, wherein said one or more excipient is added for the lyophilization process and is lyophilized.

20. The pharmaceutical composition according to claim 18, wherein said lyophilized composition is reconstituted at a point of care.

21. The pharmaceutical composition according to claim 20, wherein said lyophilized composition is reconstituted using a biocompatible, aqueous solution.

22. The pharmaceutical composition according to claim 21, wherein said biocompatible, aqueous solution is pharmaceutical-grade saline or Ringer's solution.

23. A method to remove fibrinogen from plasma according to claim 1, further comprising the step of separating said platelets from said platelet-containing solution.

24. The method according to claim 23, where said platelets are separated from said platelet-containing solution by apheresis.

25. The method according to claim 24, where said platelets are used autologously.

26. The method according to claim 24, where said platelets are used allogenically.

27. The method according to claim 24, where said platelets are outdated platelets.

28. The method according to claim 24, where said outdated platelets are supplied by blood banks.

29. The method according to claim 24, where said platelets are pooled from more than one source.

30. The method according to claim 24, where said platelets are lysed.

31. The method according to claim 30, where said platelets are lysed using sonication.

32. The method according to claim 30, where said platelets are lysed using a buffer.

33. The method according to claim 31, where said buffer is a hypotonic solution.

34. The method according to claim 24, where said platelets are frozen and when needed thawed.

35. A method to remove fibrinogen from plasma according to claim 1, wherein said fibrinogen-selective binding agent is a polyether.

36. The method to remove fibrinogen from plasma according to claim 35, wherein said polyether is an oligomer or polymer of ethylene oxide.

37. The method to remove fibrinogen from plasma according to claim 36, wherein said ethylene oxide is polyethylene glycol.

38. A method to remove fibrinogen from plasma according to claim 1, wherein said fibrinogen-selective binding agent is amorphous carbon.

39. The method to remove fibrinogen from plasma according to claim 38, wherein said amorphous carbon is diamond like carbon.

40. A method to manufacture a growth factor concentrate comprising the steps of:
collecting blood from a willing human donor;
adding an anticoagulant to said collected blood;
mixing said anticoagulant and blood well to form a uniform mixture;
separating said blood mixture until a first supernatant is completely formed;
removing the first supernatant from the blood mixture;
separating said first supernatant until a second supernatant is completely formed;
removing the second supernatant from the separated mixture;
incubating said second supernatant with a fibrinogen-selective binding agent;
separating the second supernatant containing the fibrinogen bound to said fibrinogen-selective binding agent until a third supernatant is completely formed;
suspending said third supernatant in plasma or an isotonic medium be used instead;
freezing the suspended third supernatant and plasma mixture;
thawing the frozen third supernatant and plasma mixture;
repeating the freezing and thawing steps for a select number of times; and
sterile filtering the thawed mixture to remove cellular debris until a final platelet concentrate is formed.

41. The process to manufacture a growth factor concentrate according to claim 40, wherein the freezing and thawing steps are repeated three times.

42. The process to manufacture a growth factor concentrate according to claim 40, wherein said blood is collected aseptically.

43. The process to manufacture a growth factor concentrate according to claim 40, wherein said anti-coagulant is acid citrate dextrose.

44. The process to manufacture a growth factor concentrate according to claim 40, wherein said separation steps are performed using a centrifuge without braking.

45. The process to manufacture a growth factor concentrate according to claim 44, wherein said blood and anti-coagulant mixture is separated at 100× g for a period lasting about 10 minutes into said first supernatant.

46. The process to manufacture a growth factor concentrate according to claim 40, wherein said first supernatant comprises platelet-rich plasma.

47. The process to manufacture a growth factor concentrate according to claim 46, wherein the first supernatant is transferred into a sterile conical centrifuge tubes using sterile pipettes under an isolator.

48. The process to manufacture a growth factor concentrate according to claim 47, wherein the number of platelets found in the first supernatant is determined.

49. The process to manufacture a growth factor concentrate according to claim 48, wherein the first supernatant is separated into the second supernatant using a centrifuge at about 3,300×g for 10 minutes without breaking and thereafter transferred into sterile conical centrifuge tubes.

50. The method according to claim 40, wherein said incubating step occurs overnight.

51. The method according to claim 40 or 50, wherein said incubating step occurs in a cold environment.

52. The method according to claim 51, wherein said cold environment is maintained at a temperature greater than 0° C. but less than about 10° C.

53. The method according to claim 40 or claim 51, wherein said fibrinogen-selective binding agent is a polyether.

54. The method according to claim 53, wherein said polyether is polyethylene glycol.

55. The method according to claim 54, wherein said plasma and polyethylene glycol mixture is separated into said third supernatant using a centrifuge at about 3,300×g for 10 minutes.

56. The method according to claim 40, wherein said third supernatant contains platelets.

57. The method according to claim 56, wherein said platelets are suspended in plasma to a final platelet concentration of about 1×10⁶ per ml.

58. The method according to claim 57, wherein said plasma is processed.

59. The method according to claim 58, wherein said plasma volume is generally 5 times lesser than the volume of whole blood collected from the donor.

60. The method according to claim 59, wherein said suspended platelets are frozen at about −190° to −80° C. in liquid nitrogen.

61. The method according to claim 60, wherein said frozen suspended platelets are thawed at about 25° to about 40° C.

62. The method according to claim 40, further comprising the step of dividing final thawed suspended platelet concentrate into pre-determined doses.

63. The method according to claim 40, further comprising the step of lyophilizing said final platelet concentrate into a powder.

64. A process to manufacture a growth factor concentrate comprising the steps of:
collecting blood from a willing human donor;
adding an anticoagulant to said collected blood;
mixing said anticoagulant and blood well to form a uniform mixture;
separating said blood mixture until a first supernatant is completely formed;
removing the first supernatant from the blood mixture;
separating said first supernatant until a second supernatant is completely formed;
removing the second supernatant from the separated mixture;
incubating said second supernatant with a gamma-irradiated fibrinogen-selective binding agent;
separating the second supernatant containing the fibrinogen bound to said fibrinogen-selective binding agent until a third supernatant is completely formed;
suspending said third supernatant in plasma;
freezing the suspended third supernatant and plasma mixture;
thawing the frozen third supernatant and plasma mixture; and
repeating the freezing and thawing steps a select number of times until a final platelet concentrate is formed.

65. The method according to claim 64, wherein said blood is collected via apheresis.

66. The method according to claim 65, wherein said donor has AB (Rh+) blood type.

67. The method according to claim 64, wherein said fibrinogen-selective binding agent is polyethylene glycol.

68. The method according to claim 67, wherein said second supernatant is incubated overnight.

69. The method according to claim 68, wherein said second supernatant is separated using a centrifuge at 3,300×g for 10 minutes and transferred to one or more sterile containers.

70. The method according to claim 64, further comprising a second blood sample from a willing donor.

71. The method according to claim 70, wherein said blood donor has blood type O (Rh).

72. The method according to claim 71, wherein platelets are collected from said blood sample by apheresis.

73. The method according to claim 72, further comprising the step of determining the number of platelets contained in said blood sample.

74. The method according to claim 73, wherein the third supernatant is separated using a centrifuge at about 3,300×g for 10 minutes.

75. The method according to claim 74, wherein the third supernatant is suspended in plasma to a concentration of about 1×10⁶ platelets and/or a platelet concentration that is three to eight times greater than native plasma.

76. The method according to claim 75, wherein said plasma is processed and contains thrombin and calcium chloride.

77. The method according to claim 75, wherein said plasma containing suspended platelets were frozen at about −80° C.

78. The method according to claim 77, wherein said frozen plasma containing suspended platelets is thawed at about 37° C.

79. The method according to claim 78, wherein said plasma containing suspended platelets are repeatedly frozen and thawed three times to produce a final growth factor concentrate.

80. The method according to claim 79, wherein said final growth factor concentrate is frozen for storage.

81. The method according to claim 79, comprising the additional step of lyophilizing said final growth factor concentrate.

82. The method according to claim 80, wherein said final growth factor concentrate is frozen in 5 ml aliquots.

83. Use of a growth factor concentrate prepared using the methods according to claim 6, 40 or 64 to treat a condition selected from the group consisting of chronic, non-healing cutaneous wounds, acute wounds, burns, acute external surgical wounds to the epidermis, acute surgical wounds to an internal organ, traumatic wounds, atraumatic wounds and intra-abdominal abscesses wherein said growth factor concentrate may be administered intra-dermally, sub-dermally, intra-articularly or topically.

84. The use according to claim 83, wherein said chronic, non-healing cutaneous wounds are selected from the group consisting of ischemic wounds including, but not limited to, diabetic wounds, ischemic wounds from atherosclerosis and wounds from arteriolar vasculitis; venous stasis wounds, including, but not limited to, wounds caused by post-thrombotic syndrome and post-traumatic venous stasis; pressure sores including, but not limited to, sacral decubitus ulcers, ischial decubitus ulcers and heel and malleolar decubitus ulcers; and wounds from persisting cutaneous trauma.

85. The use according to claim 83, wherein said acute wounds are selected from the group consisting of split thickness wounds including, but not limited to, skin graft donor site wounds and abrasions caused by the skin rubbing against a rough surface; and full thickness skin loss, including but not limited to, a degloving injury, traumatic skin loss and traumatic skin necrosis.

86. The use according to claim 85, wherein said abrasion is caused by an accident involving movement.

87. The use according to claim 86, wherein said movement is due to an accident involving a bicycle, scooter, moped, motorcycle, automobile, sled, skateboard, roller skates, skateboard or inline skates.

88. The use according to claim 83, wherein said burns are associated with split thickness skin graft donor site repair, acceleration of granulation tissue formation and early debridement and grafting of skin, and acceleration of re-epithelization of second-degree burns.

89. The use according to claim 83, wherein said growth factor concentrate is used to improve the cosmetic result of a skin graft by preventing chronic contracture.

90. The use according to claim 83, wherein said growth factor concentrate is used to revascularize intact skin.

91. The use according to claim 90, wherein said skin is inflicted by a condition selected from the group consisting of necrobiosis lipoidica diabeticorum, ischemia induced by radiation and pemphigus vulgaris.

92. The use according to claim 83, wherein said growth factor concentrate is used for cosmetic purposes selected from the group consisting of the treatment of androgenetic alopecia, the promotion of hair growth, the reversal of periorbital hyperpigmentation, the renewal of skin growth, the reduction of nasolabial and/or facial wrinkles, the treatment of acne and acne scars, amelioration of tennis elbow, the treatment of diabetic foot ulcers, reduction of fistulas and the reversal of age-related dermatological changes.

93. A pharmaceutical topical preparation comprising the growth factor concentrate prepared using the methods according to claim 6, 40 or 64, wherein said pharmaceutical preparation includes a slow-release biodegradable delivery system.

94. Use of the pharmaceutical topical preparation according to claim 93, wherein said preparation accelerates the rate of wound repair by shortening the lag phase.

95. The use of the pharmaceutical topical preparation according to claim 94, wherein said preparation is applied to the epidermis or to an internal organ.

96. The use of the pharmaceutical topical preparation according to claim 95, wherein said preparation is used to treat a laceration caused by a surgical procedure or a traumatic event.

97. The use of the pharmaceutical topical preparation according to claim 96, wherein said preparation is used to treat a laceration to the liver, kidney, spleen; an anastomosis of the bowel, colon or biliary tree; or atraumatic wound of the liver or spleen.

98. Use of the pharmaceutical topical preparation according to claim 93, wherein said preparation is used to accelerate the repair of human tissue due to an intra-abdominal abscess.

99. The use of the pharmaceutical topical preparation according to claim 98, wherein said abscess has be previously drained percutaneously and said preparation is injected into said drain and applied topically to the surfaces of the cavity in which the drain was placed.

100. The use of the pharmaceutical topical preparation according to claim 93, wherein said preparation is used to enhance bone grafts and accelerate soft tissue maturation in aesthetic periodontal surgery.

101. Use of a growth factor concentrate prepared using the methods according to claim 6, 40 or 64 as media for the growth or culture of cells.

102. The use according to claim 101, wherein the cells are stem cells.

103. The use according to claim 102, wherein the stem cells are mesenchymal stem cells.

104. The use according to claim 102, wherein the mesenchymal stem cells are of bone marrow origin, adipose tissue origin, umbilical cord origin, placenta origin, or dental origin.

105. The method according to claim 4, 6, 40 or 64, wherein said growth factor is selected from the group consisting of Epidermal growth factor (EGF), Vascular Endothelial growth factor (VEGF), Basic fibroblast growth factor (_{b}-FGF), Transforming growth factor-β (TGF-β) and Platelet Derived growth factor-AB (PDGF-AB).

106. The method to manufacture a growth factor concentrate according to claim 40, wherein said freezing method is selected placing the cells in an electric refrigeration unit for an extended period of time, snap-freezing, ice bath and liquid nitrogen in complete medium.
